(19) United States
(12) Patent Application Publication
(10) Pub. No.: US 2022/0133589 A1
(43) Pub. Date: May 5, 2022

(54) SYSTEMS AND METHODS FOR THERMOGRAPHIC BODY MAPPING WITH THERAPY

(71) Applicant: Sensei Wellness Holdings, Inc., Santa Monica, CA (US)

(72) Inventors: Daniel GRUNEBERG, Santa Monica, CA (US); Steven SCHKOLNE, Santa Monica, CA (US); Sara SCHKOLNE, Santa Monica, CA (US); Andy BAUCH, Santa Monica, CA (US); Mark FADIL, Santa Monica, CA (US); Brian PASS, Santa Monica, CA (US); Danny TRINH, Santa Monica, CA (US); Addison KOWALSKI, Santa Monica, CA (US)

(21) Appl. No.: 17/429,284

(22) PCT Filed: Feb. 6, 2020
(86) PCT No.: PCT/US2020/017069

§ 371 Date: Aug. 6, 2021

Related U.S. Application Data
(60) Provisional application No. 62/802,644, filed on Feb. 7, 2019.

(51) Int. Cl. A61H 37/00

(52) U.S. Cl. CPC A61H 37/00; A61H 2201/5082; A61H 2201/0142; A61H 2201/0149; A61H 2201/5097; A61H 2230/40; A61H 2230/04; A61H 2230/50; A61H 2230/20; A61H 2230/10; A61H 2201/5092

(57) ABSTRACT

Disclosed herein are systems and methods for providing a massage to an individual, comprising: sensing a parameter associated with the individual in response to the massage using a sensor; and guiding the massage based on the parameter.

### CROSS REFERENCE

This application claims the benefit of U.S. Provisional Application No. 62/802,644 filed Feb. 7, 2019, which is incorporated by reference herein in its entirety.

### BACKGROUND

Various therapy modalities exist for performing a therapy on a body of an individual including either providing or having the individual carry out, for example, massage, exercise, and/or meditation. Massage, for example, can help achieve relaxation and/or therapeutic purposes via manipulation of a subject's tissues such as rubbing, kneading, or tapping. Exercise, for example, improves physical strength, cardiovascular health, flexibility, and balance. Meditation, for example, promotes relaxation of the body with optimized breathing as well as improved awareness.

People may use massage, exercise, and/or meditation for a variety of health-related purposes, including but not limited to relief of pain, reduction of stress, rehabilitation of sports injuries, increased relaxation, addressing of anxiety and depression, and aiding in general wellness.

### SUMMARY OF THE INVENTION

Disclosed herein are methods, systems, and software for use in providing a therapy to an individual, monitoring an individual, or directing an individual to perform a therapy on themselves. Typically, a therapy as described herein is intended to improve the health of a body and/or mind of an individual. Non-limiting examples of therapy as described herein include massage, exercise, and/or meditation.

In some embodiments, the methods, systems, and software described herein are advantageous in that they provide quantitative feedback or evaluation of a therapy such as, for example, a massage, an exercise, and/or meditation. In some embodiments, such quantitative feedback or evaluation can be provided conveniently and efficiently before, during, and/or after therapy is administered. In some embodiments, the methods, systems, and software described herein can be used to allow therapy to be modified or improved based on quantitative feedback, thereby elevating the health-benefits that traditional therapies such as, for example massage, exercise, and/or meditation may provide to individuals. In some embodiments, the methods, systems, and software described herein can be used to track progress of a therapeutic treatment, a health benefit, one or more metrics of an individual's health or mindset, or any combination thereof. In some embodiments, the methods, systems, and software described herein can be used develop specific routines or specific recipes or a variety of implementations of a therapy, each that may target a different outcome, such as stress relief, reduction of blood pressure, pain relief or others.

### Quantitative Massage

For example, while it is estimated that millions of people around the world receive massage regularly, the traditional massage process does not include a data guided feedback mechanism for optimizing and enhancing the massage being delivered. Currently, with respect to traditional massage, there have been very limited tools or methods for measuring the effectiveness or quality of therapies. In general, the traditional massage process does not utilize sophisticated technology, sensors, or computing devices. Surveys or questionnaires tend to be the most common tools that are used to evaluate the quality of therapies traditionally. However, the answers to surveys and questionnaires can be objective or non-responsive and may not represent the accurate effectiveness of therapies. Evaluation of the effect of massage(s) by medical professionals can be more accurate, but it can also be costly and associated with delays. Thus, there is a significant unmet need for accurate, reliable, and convenient tools or methods for measuring the quality or effectiveness of massage(s). A thermographic map, in some embodiments, shows a representation of blood flow to or in a tissue of a body or a portion of a body of an individual, which, in some embodiments, is further used to analyze blood flow and oxygenation of tissue. In some embodiments, the blood flow data from a thermographic map is integrated with other parameters including, for example, vital signs (for example blood pressure, heart rate, pupil dilation, body temperature, oxygen levels, and others), and/or environmental parameters. A thermographic map may be combined with one or more imaging methods. Quantitative information may be obtained from a thermographic map, one or more imaging methods, or any combination thereof. One or more imaging methods may comprise ultrasound, CT scan, an magnetic resonance imaging scan, an radiography, an elastography, a photoacoustic imaging, a tomography, an echocardiography, or any combination thereof. Images from a thermographic map and images acquired from a second imaging modality may be (i) acquired concurrently or consequently; (ii) overlaid in a single image or viewed separately, (iii) or any combination thereof.

### Quantitative Exercise

Exercise and movement are widely used means of therapy and personal health improvement and maintenance, and in some cases exercise and structured movement (e.g., physical therapy, dance) are aided and/or guided by monitoring devices. There is, however, an absence of a holistic monitoring approach such as the one described herein wherein, as in some embodiments, thermographic mapping is combined with vital sign monitoring information (for example, blood pressure, heart rate, pupil dilation, body temperature, oxygen levels, and others) and/or other monitored parameters to provide feedback used, in some embodiments, to analyze exercise and movement and in some embodiments offer suggestions for modifications. For example, blood flow and oxygen delivery in particular are important to gaining optimal exercise benefits and performance, and in some embodiments described herein a thermographic map is generated of a body of an individual or a portion their body. A thermographic map, in some embodiments, shows a representation of blood flow to or in a tissue of a body or a portion of a body of an individual, which, in some embodiments, is further used to analyze blood flow and oxygenation of tissue. In some embodiments, the blood flow data from a thermographic map is integrated with other parameters including, for example, vital signs, and/or environmental parameters. Such an approach, as described herein, is holistic, and different from traditional approaches, in that, for example, it integrates traditional vital sign monitoring as well as other traditional (and non-traditional) monitoring modalities to generate advanced analytics regarding complex body metrics such as, for example, blood flow and oxygen delivery. A thermographic map may be combined with one or more imaging methods. Quantitative information may be obtained from a thermographic map, one or more imaging methods, or any combination thereof. One or more imaging methods may comprise ultrasound, CT scan, an magnetic resonance imaging scan, an radiography, an elastography, a photoacoustic imaging, a tomography, an echocardiography, or any combination thereof. Images from a thermographic map and images acquired from a second imaging modality may be (i) acquired concurrently or consequently; (ii) overlaid in a single image or viewed separately, (iii) or any combination thereof.

### Quantitative Meditation

The traditional meditation process does not include a data guided feedback mechanism for optimizing and enhancing the meditation being delivered. In some embodiments of systems, devices, software, and methods described herein, sensed parameters are used to analyze and/or modify or suggest a modification of the meditation experience. A thermographic map, in some embodiments, shows a representation of blood flow to or in a tissue of a body or a portion of a body of an individual, which, in some embodiments, is further used to analyze blood flow and oxygenation of tissue. In some embodiments, the blood flow data from a thermographic map is integrated with other parameters including, for example, vital signs, and/or environmental parameters. A thermographic map may be combined with one or more imaging methods. Quantitative information may be obtained from a thermographic map, one or more imaging methods, or any combination thereof. One or more imaging methods may comprise ultrasound, CT scan, an magnetic resonance imaging scan, an radiography, an elastography, a photoacoustic imaging, a tomography, an echocardiography, or any combination thereof. Images from a thermographic map and images acquired from a second imaging modality may be (i) acquired concurrently or consequently; (ii) overlaid in a single image or viewed separately, (iii) or any combination thereof.

### Innovative Methods, Systems, and Software

Described herein are methods, systems, and software for quantitatively guiding an administration of a therapy such as, for example, massage, exercise, and/or meditation to an individual (administered by another individual or the individual themselves) or monitoring an individual. Quantitative guidance, in some embodiments, is based on sensed parametric data sensed from the individual and/or other individuals.

In some embodiments, parametric data used by the systems, devices, software, and methods described herein is sensed from an individual receiving or undergoing a therapy such as massage, exercise, and/or meditation. In some embodiments, parametric data used by the systems, devices, software, and methods described herein is sensed from individuals other than an individual who is undergoing a therapy such as a massage, exercise, and/or meditation. In some embodiments, sensed data is processed and determines a type of guidance to be provided to a therapy provider or to a person providing therapy to themselves. In this way, the therapy being provided is guided by data.

Disclosed herein are methods, systems, and software for use in providing a massage to an individual. In some embodiments, the methods, systems, and software described herein are advantageous in that they provide quantitative feedback or evaluation of a therapy. In some embodiments, such quantitative feedback or evaluation can be provided conveniently and efficiently before, during, and/or after a therapy. In some embodiments, the methods, systems, and software herein can be used to allow a therapy such as a massage, exercise, and/or meditation to be modified or improved based on the quantitative feedback(s), thereby more effectively providing the health-benefits that traditional therapies provide to individuals.

In one aspect, disclosed herein is a method for providing a massage to an individual, the method comprising: (a) sensing a parameter associated with the individual in response to the massage using a sensor; and (b) guiding the massage based on the parameter. In some embodiments, the parameter comprises a movement of a portion of a body of the individual. In some embodiments, the sensor comprises an infrared light sensor. In some embodiments, the infrared light sensor is operably coupled with an infrared light source that is configured to project an infrared light towards the individual, and wherein the infrared light sensor is configured to sense the infrared light when the infrared light is reflected from the individual. In some embodiments, the parameter comprises a temperature of a portion of a body of the individual. In some embodiments, the sensor comprises a thermographic camera. In some embodiments, the sensor comprises a temperature probe or pad. In some embodiments, the parameter comprises a vital sign of the individual. In some embodiments, the sensor comprises a heart rate sensor. In some embodiments, the sensor comprises a blood pressure cuff. In some embodiments, the sensor comprises an spO₂ sensor. In some embodiments, the parameter comprises an electrocardiogram (ECG) of the individual. In some embodiments, the sensor comprises at least one ECG electrode. In some embodiments, the parameter comprises an electroencephalogram (EEG). In some embodiments, the sensor comprises at least one EEG sensor. In some embodiments, step (b) comprises indicating to a massage provider to modify the massage. In some embodiments, the parameter is sensed in response to the massage to a first portion of a body of the individual and wherein step (b) comprises indicating to a massage provider to modify the massage to the first portion of the body. In some embodiments, indicating to a massage provider to modify the massage to the first portion of the body comprises indicating to the massage provider to apply more pressure to the first portion of the body. In some embodiments, indicating to a massage provider to modify the massage to the first portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to the first portion of the body. In some embodiments, the parameter comprises a movement of the first portion of the body of the individual. In some embodiments, the parameter comprises a thermal reading of the first portion of the body. In some embodiments, the parameter is sensed in response to the massage to a first portion of a body of the individual and wherein the parameter comprises at least one of: a respiration rate, an oxygenation, a heart rate, a heart rhythm, a blood pressure, or a brain function. In some embodiments, the parameter is sensed in response to the massage to a first portion of a body of the individual and wherein step (b) comprises indicating to a massage provider to modify the massage to a second portion of the body. In some embodiments, indicating to a massage provider to modify the massage to the second portion of the body comprises indicating to the massage provider to apply more pressure to the second portion of the body. In some embodiments, indicating to a massage provider to modify the massage to the second portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to a second portion of the body. In some embodiments, the parameter comprises a movement of the second portion of the body of the individual. In some embodiments, the parameter comprises a thermal reading of the second portion of the body. In some embodiments, the parameter is received as an input to a machine learning algorithm configured to output a guidance. In some embodiments, step (b) comprises presenting the guidance to a massage provider.

In another aspect, disclosed herein is a system for use in providing a massage to an individual, the system comprising: a surface upon which the individual lies or sits; a first sensor coupled to the surface and configured to sense a parameter associated with the individual when the individual lies or sits on the surface; and a second sensor positioned to sense a signal emitted by or reflected by a portion of the individual when the individual lies or sits on the surface. In some embodiments, the surface comprises a massage table. In some embodiments, the surface comprises a massage chair. In some embodiments, the first sensor is positioned on the surface so that it contacts the individual when the individual lies or sits on the surface. In some embodiments, the first sensor comprises a vital sign sensor. In some embodiments, the first sensor comprises a heart rate sensor. In some embodiments, the first sensor comprises a blood pressure sensor. In some embodiments, the first sensor comprises an spO₂ sensor. In some embodiments, the first sensor comprises an ECG sensor. In some embodiments, the first sensor comprises an EEG sensor. In some embodiments, the second sensor comprises an infrared sensor. In some embodiments, the infrared sensor is a component of a thermogenic camera. In some embodiments, the second sensor is configured to detect motion. In some embodiments, the second sensor is configured to sense energy emitted or reflected from the individual. In some embodiments, the system comprises at least one of: an infrared emitter, an ultrasound emitter, or a radiofrequency emitter. In some embodiments, the system comprises a non-transitory computer readable medium configured to receive the parameter and the signal and output a guidance to a massage provider. In some embodiments, the guidance is to modify a massage to a portion of a body of an individual. In some embodiments, the guidance is to apply more pressure to the portion. In some embodiments, the guidance is to apply less pressure to the portion. In some embodiments, the guidance is to apply additional massage to the portion. In some embodiments, the guidance is to cease massage to the first portion. In some embodiments, the system comprises a remote server configured to receive and analyze the parameter and the signal as well as a massage provider application configured to communicate with the remote server. In some embodiments, the system comprises a massage recipient application configured to communicate with the remote server.

In yet another aspect, disclosed herein is a non-transitory computer readable medium comprising a computer program including instructions that cause a processor to: receive a parameter of an individual sensed with a parameter sensor; and determine a guidance comprising a modification of a massage delivered by a massage provider to the individual. In some embodiments, the parameter comprises a movement of a portion of a body of the individual. In some embodiments, the sensor comprises an infrared light sensor. In some embodiments, the infrared light sensor is operably coupled with an infrared light source that is configured to project an infrared light towards the individual, and wherein the infrared light sensor is configured to sense the infrared light when the infrared light is reflected from the individual. In some embodiments, the parameter comprises a temperature of a portion of a body of the individual. In some embodiments, the sensor comprises a thermographic camera. In some embodiments, the sensor comprises a temperature probe or pad. In some embodiments, the parameter comprises a vital sign of the individual. In some embodiments, the sensor comprises a heart rate sensor. In some embodiments, the sensor comprises a blood pressure cuff. In some embodiments, the sensor comprises an spO₂ sensor. In some embodiments, the parameter comprises an electrocardiogram (ECG) of the individual. In some embodiments, the sensor comprises at least one ECG electrode. In some embodiments, the parameter comprises an electroencephalogram (EEG). In some embodiments, the sensor comprises at least one EEG sensor. In some embodiments, the parameter is sensed in response to the massage to a first portion of a body of the individual and wherein the guidance comprises indicating to a massage provider to modify the massage to the first portion of the body. In some embodiments, indicating to a massage provider to modify the massage to the first portion of the body comprises indicating to the massage provider to apply more pressure to the first portion of the body. In some embodiments, indicating to a massage provider to modify the massage to the first portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the guidance relates to the first portion of the body. In some embodiments, the parameter comprises a movement of the first portion of the body of the individual. In some embodiments, the parameter comprises a thermal reading of the first portion of the body. In some embodiments, the parameter is sensed in response to the massage to a first portion of a body of the individual and wherein the parameter comprises at least one of: a respiration rate, an oxygenation, a heart rate, a heart rhythm, a blood pressure, or a brain function. In some embodiments, the parameter is sensed in response to the massage to a first portion of a body of the individual and wherein the guidance comprises indicating to a massage provider to modify the massage to a second portion of the body. In some embodiments, indicating to a massage provider to modify the massage to the second portion of the body comprises indicating to the massage provider to apply more pressure to the second portion of the body. In some embodiments, indicating to a massage provider to modify the massage to the second portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to a second portion of the body. In some embodiments, the parameter comprises a movement of the second portion of the body of the individual. In some embodiments, the parameter comprises a thermal reading of the second portion of the body.

In yet another aspect, disclosed herein is a method for guiding meditation, the method comprising: (a) sensing a parameter associated with the individual while the individual is meditating using a sensor; and (b) providing guidance to the individual based on the parameter. In some embodiments, the parameter comprises a movement of a portion of a body of the individual. In some embodiments, the sensor comprises an infrared light sensor. In some embodiments, the infrared light sensor is operably coupled with an infrared light source that is configured to project an infrared light towards the individual, and wherein the infrared light sensor is configured to sense the infrared light when the infrared light is reflected from the individual. In some embodiments, the parameter comprises a temperature of a portion of a body of the individual. In some embodiments, the sensor comprises a thermographic camera. In some embodiments, the sensor comprises a temperature probe or pad. In some embodiments, the parameter comprises a vital sign of the individual. In some embodiments, the sensor comprises a heart rate sensor. In some embodiments, the sensor comprises a blood pressure cuff. In some embodiments, the sensor comprises an spO2sp02 sensor. In some embodiments, the parameter comprises an electrocardiogram (ECG) of the individual. In some embodiments, the sensor comprises at least one ECG electrode. In some embodiments, the parameter comprises an electroencephalogram (EEG). In some embodiments, the sensor comprises at least one EEG sensor. In some embodiments, step (b) comprises indicating the individual to modify the meditation. In some embodiments, indicating to the individual to modify the meditation comprises an indication for the individual to modify their breathing. In some embodiments, indicating to the individual to modify the meditation comprises an indication for the individual to increase their concentration. In some embodiments, indicating to the individual to modify the meditation comprises an indication for the individual to cease the meditation.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting exemplary process flow of the method for providing a massage to an individual;

FIG. 2 shows a non-limiting exemplary process flow of the method for providing a massage to an individual;

FIG. 3 shows a non-limiting exemplary schematic diagram of the system for providing a massage to an individual;

FIG. 4 shows a non-limiting schematic diagram of a digital processing device; in this case, a device with one or more CPUs, a memory, a communication interface, and a display;

FIG. 5 shows a non-limiting schematic diagram of a web/mobile application provision system; in this case, a system providing browser-based and/or native mobile user interfaces;

FIG. 6 shows a non-limiting schematic diagram of a cloud-based web/mobile application provision system; in this case, a system comprising an elastically load balanced, auto-scaling web server and application server resources as well synchronously replicated databases;

FIG. 7 shows an exemplary standing configuration where an individual is in a standing position on a surface;

FIG. 8 shows an exemplary table configuration;

FIG. 9 shows an exemplary wiring diagram;

FIG. 10 shows an exemplary flow diagram of a therapy session;

FIG. 11 shows an exemplary flow diagram of a camera; and

FIG. 12 shows an exemplary flow diagram of a gallery.

### DETAILED DESCRIPTION OF THE INVENTION

Described herein, in certain embodiments, are methods, systems, and software for guiding a therapy provided to an individual including therapies such as, for example, massage, exercise, and/or meditation to an individual. In general, a therapy as described herein comprises a therapy to a body or a mind of an individual. Non-limiting examples of therapy as described herein include massage, acupuncture, cryotherapy, exercise, running, weight training, swimming, cycling, competitive sports, meditation, hypnosis, and combinations thereof.

Described herein, in certain embodiments, are methods, systems, and software for monitoring an individual. In some embodiments, an individual is monitored using a thermographic body mapping technique. For example, a thermographic body map of an individual may be generated while an individual is sleeping and the map may be further used to, for example, analyze the sleep quality of the individual and/or the effect the sleep has on the body of the individual. That is, in some embodiments, a thermogenic body map of an individual may be used to monitor an individual over a period of time and the map may be, in some embodiments, used to analyze one or more of the individual, a behavior engaged in by the individual, and/or the effect of a behavior engaged in by the individual on the body of the individual.

Disclosed herein, in some embodiments, are methods for guiding an application of a therapy to an individual comprises: (a) sensing a parameter associated with the individual in response to a therapy using a sensor; and (b) guiding the therapy based on the parameter. In some embodiments, the parameter comprises a movement of a portion of a body of the individual. In some embodiments, the sensor comprises an infrared light sensor. In some embodiments, the infrared light sensor is operably coupled with an infrared light source that is configured to project an infrared light towards the individual, and wherein the infrared light sensor is configured to sense the infrared light when the infrared light is reflected from the individual. In some embodiments, the parameter comprises a temperature of a portion of a body of the individual. In some embodiments, the sensor comprises a thermographic camera. In some embodiments, the sensor comprises a temperature probe or pad. In some embodiments, the parameter comprises a vital sign of the individual. In some embodiments, the sensor comprises a heart rate sensor. In some embodiments, the sensor comprises a blood pressure cuff. In some embodiments, the sensor comprises a spO₂ sensor. In some embodiments, the parameter comprises an electrocardiogram (ECG) of the individual. In some embodiments, the sensor comprises at least one ECG electrode. In some embodiments, the parameter comprises an electroencephalogram (EEG). In some embodiments, the sensor comprises at least one EEG sensor. In some embodiments, step (b) comprises indicating to a massage provider to modify the massage. In some embodiments, the parameter is sensed in response to the massage to a first portion of a body of the individual and wherein step (b) comprises indicating to a massage provider to modify the massage to the first portion of the body. In some embodiments, indicating to a massage provider to modify the massage to the first portion of the body comprises indicating to the massage provider to apply more pressure to the first portion of the body. In some embodiments, indicating to a massage provider to modify the massage to the first portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to the first portion of the body. In some embodiments, the parameter comprises a movement of the first portion of the body of the individual. In some embodiments, the parameter comprises a thermal reading of the first portion of the body. In some embodiments, the parameter is sensed in response to the massage to a first portion of a body of the individual and wherein the parameter comprises at least one of: a respiration rate, an oxygenation, a heart rate, a heart rhythm, a blood pressure, or a brain function. In some embodiments, the parameter is sensed in response to the massage to a first portion of a body of the individual and wherein step (b) comprises indicating to a massage provider to modify the massage to a second portion of the body. In some embodiments, indicating to a massage provider to modify the massage to the second portion of the body comprises indicating to the massage provider to apply more pressure to the second portion of the body. In some embodiments, indicating to a massage provider to modify the massage to the second portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to a second portion of the body. In some embodiments, the parameter comprises a movement of the second portion of the body of the individual. In some embodiments, the parameter comprises a thermal reading of the second portion of the body. In some embodiments, the parameter is received as an input to a machine learning algorithm configured to output a guidance. In some embodiments, step (b) comprises presenting the guidance to a massage provider.

Disclosed herein, in some embodiments, are systems for use in guiding an application of a therapy to an individual, the system comprising: a surface upon which the individual lies or sits; a first sensor coupled to the surface and configured to sense a parameter associated with the individual when the individual lies or sits on the surface; and a second sensor positioned to sense a signal emitted by or reflected by a portion of the individual when the individual lies or sits on the surface. In some embodiments, the surface comprises a massage table. In some embodiments, the surface comprises a massage chair. In some embodiments, the first sensor is positioned on the surface so that it contacts the individual when the individual lies or sits on the surface. In some embodiments, the first sensor comprises a vital sign sensor. In some embodiments, the first sensor comprises a heart rate sensor. In some embodiments, the first sensor comprises a blood pressure sensor. In some embodiments, the first sensor comprises a spO₂ sensor. In some embodiments, the first sensor comprises an ECG sensor. In some embodiments, the first sensor comprises an EEG sensor. In some embodiments, the second sensor comprises an infrared sensor. In some embodiments, the infrared sensor is a component of a thermogenic camera. In some embodiments, the second sensor is configured to detect motion. In some embodiments, the second sensor is configured to sense energy emitted or reflected from the individual. In some embodiments, the system comprises at least one of: an infrared emitter, an ultrasound emitter, or a radiofrequency emitter. In some embodiments, the system comprises a non-transitory computer readable medium configured to receive the parameter and the signal and output a guidance to a massage provider. In some embodiments, the guidance is to modify a massage to a portion of a body of an individual. In some embodiments, the guidance is to apply more pressure to the portion. In some embodiments, the guidance is to apply less pressure to the portion. In some embodiments, the guidance is to apply additional massage to the portion. In some embodiments, the guidance is to cease massage to the first portion. In some embodiments, the system comprises a remote server configured to receive and analyze the parameter and the signal as well as a massage provider application configured to communicate with the remote server. In some embodiments, the system comprises a massage recipient application configured to communicate with the remote server. In some embodiments, one or more sensors are configured to sense one or more parameters while an individual is standing and not in contact with a table or chair surface.

Disclosed herein, in some embodiments, are non-transitory computer readable media comprising a computer program including instructions that cause a processor to: receive a parameter of an individual sensed with a parameter sensor; and determine a guidance comprising a modification of a massage delivered by a massage provider to the individual. In some embodiments, the parameter comprises a movement of a portion of a body of the individual. In some embodiments, the sensor comprises an infrared light sensor. In some embodiments, the infrared light sensor is operably coupled with an infrared light source that is configured to project an infrared light towards the individual, and wherein the infrared light sensor is configured to sense the infrared light when the infrared light is reflected from the individual. In some embodiments, the parameter comprises a temperature of a portion of a body of the individual. In some embodiments, the sensor comprises a thermographic camera. In some embodiments, the sensor comprises a temperature probe or pad. In some embodiments, the parameter comprises a vital sign of the individual. In some embodiments, the sensor comprises a heart rate sensor. In some embodiments, the sensor comprises a blood pressure cuff. In some embodiments, the sensor comprises a spO₂ sensor. In some embodiments, the parameter comprises an electrocardiogram (ECG) of the individual. In some embodiments, the sensor comprises at least one ECG electrode. In some embodiments, the parameter comprises an electroencephalogram (EEG). In some embodiments, the sensor comprises at least one EEG sensor. In some embodiments, the parameter is sensed in response to the massage to a first portion of a body of the individual and wherein the guidance comprises indicating to a massage provider to modify the massage to the first portion of the body. In some embodiments, indicating to a massage provider to modify the massage to the first portion of the body comprises indicating to the massage provider to apply more pressure to the first portion of the body. In some embodiments, indicating to a massage provider to modify the massage to the first portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the guidance relates to the first portion of the body. In some embodiments, the parameter comprises a movement of the first portion of the body of the individual. In some embodiments, the parameter comprises a thermal reading of the first portion of the body. In some embodiments, the parameter is sensed in response to the massage to a first portion of a body of the individual and wherein the parameter comprises at least one of: a respiration rate, an oxygenation, a heart rate, a heart rhythm, a blood pressure, or a brain function. In some embodiments, the parameter is sensed in response to the massage to a first portion of a body of the individual and wherein the guidance comprises indicating to a massage provider to modify the massage to a second portion of the body. In some embodiments, indicating to a massage provider to modify the massage to the second portion of the body comprises indicating to the massage provider to apply more pressure to the second portion of the body. In some embodiments, indicating to a massage provider to modify the massage to the second portion of the body comprises indicating to the massage provider to apply less pressure to the first portion of the body. In some embodiments, indicating to a massage provider to modify the massage to the first portion of the body comprises indicating to the massage provider to apply additional massage to the first portion of the body. In some embodiments, indicating to a massage provider to modify the massage to the first portion of the body comprises indicating to the massage provider to cease massage to the first portion of the body. In some embodiments, the parameter is sensed in response to the massage to a first portion of a body of the individual and wherein the parameter relates to a second portion of the body. In some embodiments, the parameter comprises a movement of the second portion of the body of the individual. In some embodiments, the parameter comprises a thermal reading of the second portion of the body.

### Certain Terms

Unless otherwise defined, all technical terms used herein have the same meaning as commonly understood by one of ordinary skill in the art to which this invention belongs.

As used herein, a “therapy” includes any process intended to improve the health of a body and/or mind of an individual including but not limited to massage, exercise, and/or meditation. Therapy may include massage, exercise, meditation, reflexology, aromatherapy, acupressure, acupuncture, homeopathy, Ayurveda, balneotherapy, naturopathy, biofeedback, moxibustion, cupping, herbal treatment, or any combination thereof. Therapy may include psychotherapy, rest, hypnosis, or meditation.

As used herein “exercise” includes traditional forms of exercise as well as other types of movements including but not limited to all forms of aerobic exercise, stretching, yoga, Pilates, and physical therapy. Exercise may include stretching, yoga, pilates, physical therapy, breathing, dancing, running, swimming, walking, biking, or any combination thereof.

As used herein, the singular forms “a,” “an,” and “the” include plural references unless the context clearly dictates otherwise. Any reference to “or” herein is intended to encompass “and/or” unless otherwise stated.

As used herein, the term “about ” refers to an amount that is near the stated amount by about 10%, 5%, or 1%, including increments therein.

As used herein, the term “massage” is equivalent to a massage session which can include different portions. A massage may comprise a Swedish massage, a hot stone massage, an aromatherapy massage, a deep tissue massage, a sport massage, a trigger point massage, a reflexology massage, a Shiatsu massage, a Thai massage, a prenatal massage, or any combination thereof. In some embodiments, the massage herein includes a time duration, e.g., at least about 5 minutes, at least about 10 minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, at least about 60 minutes or any other time duration. In some embodiments, a portion of a massage session can be from about 1% to about 99% of the massage session.

In some embodiments, the massage provider is the individual receiving the massage. In some embodiments, the massage provider is an individual. In some embodiments, the massager provider is a trained professional. In some embodiments, the massage provider is any device that is configured to provide massage session(s) to an individual, e.g, a robotic arm. In some embodiments, the massage provider can be a massage table, a massage chair, a robotic arm, or a combination thereof.

### Therapy—Including Quantitative Massage

In some embodiments, disclosed herein are therapies that use sensing of parameter(s) of a human subject to guide the therapy based on the parameter. Massage is one type of therapy described herein for which exemplary embodiments are described. It should be understood that systems, devices, methods, and software described herein are suitable for use with other therapies including, for example, exercise and meditation as well.

In some embodiments, the parameters of the subject being sensed can include any biological or physiological parameters. In some embodiments, the parameters can be sensed using one or more sensors. For example, 2, 3, 4, 5, 6, 7, 8, 9, 10 sensors or more may be employed to measure the parameters. In some embodiments, the therapy provider can evaluate the values of the sensed parameter(s) and plan out schemes of future therapies or modifications to an ongoing therapy. In some embodiments, the values of the sensed parameter(s) can be evaluated automatically, and future therapies can be designed automatically using the parameters. As such, the effectiveness or quality of the therapy can be accurately and reliably optimized.

In some embodiments, disclosed herein are methods for providing a therapy to an individual. In some embodiments, the methods herein include sensing one or more parameters associated with an individual using one or more sensors. In some embodiments, one or more parameters herein are in response to a number of previously performed therapy(s). In some embodiments, one or more parameters herein are sensed as a baseline before any therapy(s) are performed. In some embodiments, the one or more parameters are sensed during a therapy as an evaluation when at least a portion of the therapy is going to be performed. In some embodiments, the one or more parameters are sensed during a therapy for monitoring the status of the individual. In some embodiments, the one or more parameters are sensed after a therapy as an evaluation of the past therapy(s) and as a reference for performing future therapy(s). A parameter may be sensed continuously. A parameter may be sensed intermittently, such as before a therapy, during a therapy, and after a therapy. A measured value of a parameter may be recorded. The measured value may be recorded continuously. A measured value of a parameter may be measured intermittently. A measured value of a parameter may be stored in a database.

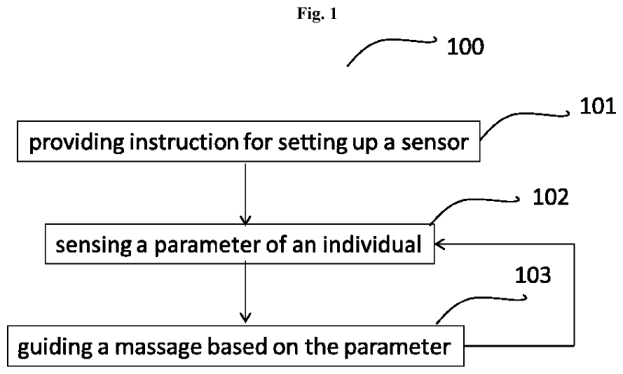

Referring to FIG. 1, in a particular embodiment, the method for providing a massage to an individual 100 may include an operation that provides instructions for the massage provider to set up one or more sensors 101 before a massage session starts. In the same embodiment, subsequent to providing set-up instructions, the method includes sensing a parameter of an individual 102, e.g., before a massage session starts or during a massage. Subsequently, the parameter is used to guide at least a portion of a massage 103. In some embodiments, the method can stop after operation 103 is performed. In some embodiments, after the massage or at least a portion of the massage is performed, operations 102 and 103 are repeated until a predetermined condition is met.

In some embodiments, the pre-determined condition can be set by the individual, the massage provider, or a computer program automatically. For example, the pre-determined condition can be a time duration for the massage session. As another example, the pre-determined condition may be a percentage of change of one or more sensed parameters. As yet another example, the pre-determined condition can be a variation in a vital sign of the individual.

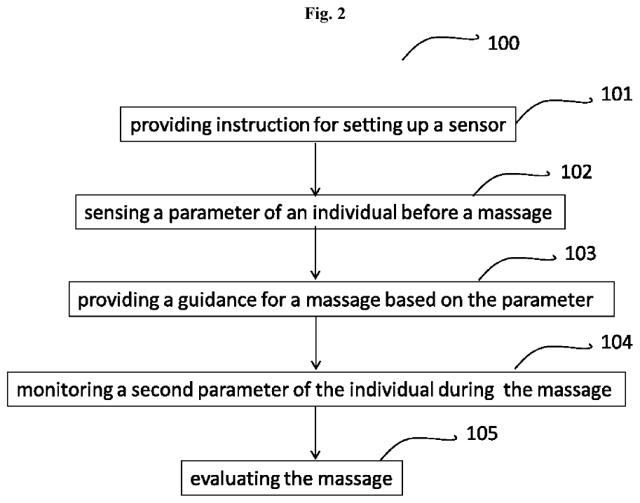

Referring to FIG. 2, in a particular embodiment, the method for providing a massage to an individual 100 may include an operation that provides instruction for the massage provider to set up one or more sensors 101 before a massage session starts. In the same embodiment, subsequent to providing set-up instructions, the method includes sensing a parameter of an individual 102 before a massage session starts. Subsequently, the parameter is presented or otherwise input to a system disclosed herein so that the system generates and provides a guidance to the massage provider for performing a massage on the individual based on the sensed parameter 103. The massage provider can perform the massage based on the guidance. Before performing the massage, the system herein can be used to provide instructions to the massage provider or a user for setting up one or more sensors for monitoring a second parameter of the individual. During at least a portion of the massage, the method includes monitoring the second parameter of the individual 104. In some embodiments, the second parameter is monitored at least in part for ensuring the wellness or status of the individual during the massage, especially when the massage is guided based on the first parameter. In some embodiments, the second parameter is monitored at least in part as a measure of an effect of the massage. For example, the second parameter can be a respiration rate, heart rate, a temperature, or blood pressure of the individual. After the massage session is completed, the method herein includes an operation that evaluates the massage 105. In some embodiments, the massage can be evaluated using a follow-up massage or questionnaire that can be filled out by the individual. In some embodiments, the massage can be evaluated by measuring or sensing one or more parameters of the individual. In further embodiments, the one or more parameters sensed before and after massage can be compared and/or the difference of the one or more parameters can be measured. In some embodiments, monitoring of a second parameter of the individual may occur, such as during the massage 104. A third parameter, a fourth parameter or additional parameters may be monitored during the massage. In some embodiments, an evaluation of the massage may occur, such as before the massage, during the massage, after completion of the massage, or any combination 105. The evaluation may be quantitative.

As an example, the massage provider can have the guest or individual lie on the massage table face up through a five minute session of guided meditation/relaxation, then take anterior thermograms of the guest. Afterwards, the guest can turn over to be face down and go through another 5 minute session of guided meditation, and then posterior thermograms can be taken. In this particular embodiment, sensors that work compatibly with a massage table can be used to send live heart rate information wirelessly to a digital processing device. Such heart rate information can be monitored before, during, and/or after massage. A reduced heart rate associated with increased relaxation can be expected during or after massage, and an increased heart rate can be indicative of increased pain while the massage provider is working on problematic areas. After the thermal images have been taken and reviewed there are several things that can be considered by the massage provider or automatically analyzed by a computer program. For example, in the acquired thermograms, the symmetry of the human body is helpful in finding thermal abnormalities. The massage provider or a computer algorithm can search for areas of asymmetry and examine if the areas match locations of pain as the patient has described. If there is a match and/or if there is abnormality in the thermograms, e.g., asymmetry in symmetrical portions of the body, a guidance can be provided to the massage provider to focus on the match or abnormal areas in the themograms.

In some embodiments, the parameter is combined with the patient or the individual's input to generate the guidance for at least a portion of the massage. As a nonlimiting example, the asymmetry in symmetrical portions of the body may be provided and superimposed with the individual's drawing of pain location(s) and pain levels to determine if certain areas of the body may need more intensive massage or not.

In some embodiments, sensing of the parameter(s) may result in a numerical value with or without unit. In some embodiments, sensing of the parameter(s) may result in multiple values that can be numerical, e.g., sensing result can be an image of a certain portion of the individual. In some embodiments, sensing may result in values in more than one parameter.

In some embodiments, the sensor comprises an infrared light sensor. In some embodiments, the infrared light sensor is operably coupled with an infrared light source that is configured to project an infrared light towards the individual, and wherein the infrared light sensor is configured to sense said infrared light when said infrared light is reflected from the individual. In some embodiments, the parameter comprises a temperature of a portion of a body of said individual. The sensor may comprise a thermographic camera, a temperature probe, and/or pad. In some embodiments, the parameter comprises a vital sign of an individual. The sensor can be a heart rate sensor, a blood pressure cuff, a spO₂ sensor, etc. In some embodiments, the parameter(s) include an electrocardiogram (ECG) of said individual. The sensor may comprise at least one ECG electrode. In some embodiments, the parameter(s) comprises an electroencephalogram (EEG), and the sensor comprises at least one EEG sensor.

In some embodiments, the parameter includes a movement of a portion of the body of the individual. In some embodiments, the parameter includes one or more of: a respiration rate, an oxygenation, a heart rate, a heart rhythm, a blood pressure, a blood glucose level, a muscle action potential, or a brain function. In some embodiments, the parameter includes a thermal reading.

In some embodiments, the sensor is placed on at least a portion of the body of the individual. For example, an EEG sensor or one or more EEG leads are attached to the chest of the individual. As another example, a blood oxygen sensor can be clipped on a finger of the individual. In some embodiments, the sensor is in contact with at least a portion of the body of the individual. As non-limiting examples, the sensor can be placed on the massage table, the massage provider, a piece of clothing, or any other objects that the individual may contact. In some embodiments, the sensor is not in contact with the individual. As a non-limiting example, an infrared camera can be placed within a distance to the individual without obstacles to measure reflected infrared signals from the individual.

In some embodiments, the sensor herein includes but is not limited to one or more of: a temperature sensor, a humidity sensor, an electrical impedance sensor, an acoustic impedance sensor, an electromyography (EMG) sensor, an oxygen sensor, a pH sensor, an optical sensor, an ultrasound sensor, a glucose sensor, a biomarker sensor, a heart rate monitor, a respirometer, an electrolyte sensor, a blood pressure sensor, an EEG sensor, an ECG sensor, a body hydration sensor, a carbon dioxide sensor, a carbon monoxide sensor, a blood alcohol sensor, and a Geiger counter.

In some embodiments, the sensor herein is set-up so that it may minimize the discomfort it may cause the individual. In some embodiments, the sensor herein is set-up so that the interference to the individual's privacy is minimized. In some embodiments, the individual may be provided with options as how the sensor is set-up. As an example, the individual may not want any sensor to be attached to his body, and he can select the sensor that is embedded on the massage table and can contact his body while he lies on the massage table. A sensor may contact a surface of an individual's body. A sensor may be proximal a surface of an individual's body. A sensor may be attached to an individual's body. A sensor may be configured as part of a system as described herein, such as configured as part of a massage table or massage chair. A massage table or massage chair may comprise one or more sensors. A sensor may be operated by the individual A sensor may be operated by a professional, such as a massage profession.

In some embodiments, the methods, systems, and software herein utilize one or more parameters to guide subsequent portions or sessions of massage or other therapy. In some embodiments, guiding subsequent portions or sessions of massage includes indicating to a massage provider to modify the massage to at least one portion of the body of the individual. In some embodiments, the parameter is sensed in a first portion of a body of said individual and such parameter is used as basis for modifying the massage to the same portion or a different portion of the body. For example, a parameter is sensed in one leg of the individual and based on the sensed value of the parameter, a user or a computer program can indicate to a massage provider to modify the massage of the leg such as changing the massage pressure, applying additional massage to the leg, ceasing massage to the leg, or the like. A modification may comprise one or more of: changing a pressure applied by the massage, changing a type of massage (Swedish massage, deep tissue massage, or other), changing a duration of a massage at a given location on an individual's body, changing a location on an individual's body receiving the massage, or any combination thereof. One or more parameters may include an infrared map of a portion of an individual's body, a temperature of a portion of an individual's body, a blood pressure, a heart rate, a pupil dilation, a brain wave, a moisture content on a surface of an individual's body, or any combination thereof.

### Systems for Therapy

In some embodiments, disclosed herein is a system for use in providing a therapy to an individual. In some embodiments, the system is a computer-implemented system. In some embodiments, the system includes a digital processing device. In some embodiments, the system herein includes one or more computer program or algorithm. In some embodiments, the system herein includes a database. In some embodiments, the system herein includes a surface upon which an individual may lay down or may site. In some embodiments, the surface is part of a therapy table, a therapy chair, a therapy bed, or the like. In some embodiments, the surface includes a mechanical mechanism that is configured to automatically apply a therapy to an individual that lies or sits thereon. In some embodiments, the surface may be powered or not powered. In some embodiments, the surface is not part of system herein.

In some embodiments, the surface includes one or more sensors embedded therein or attached thereon. In some embodiments, the system herein includes a first sensor coupled to the surface and configured to sense one or more parameter associated with said individual when said individual lies or sits on said surface. In some embodiments, the system herein includes a second sensor positioned to sense a signal emitted by or reflected by a portion of said individual when said individual lies or sits on said surface. The first sensor is positioned on said surface so that it can contact the individual when the individual lies or sits on said surface. In some embodiments, the first sensor comprises a vital sign sensor, a heart rate sensor, a blood pressure sensor, a spO₂ sensor, an ECG sensor, an EEG sensor, an infrared sensor, a component of a thermogenic camera.

In some embodiments, the second sensor is configured to detect motion. In some embodiments, the second sensor is a motion sensor. In some embodiments, the second sensor is configured to sense energy emitted or reflected from the individual.

In some embodiments, the system includes at least one of: an infrared emitter, an ultrasound emitter, or a radiofrequency emitter. In some embodiments, the emitter herein can be attached to the surface herein. In some embodiments, the emitter is located remotely from the surface, the sensor, or the individual when the individual lies or sits on the surface. For example, the emitter may be attached on a wall or the roof.

In some embodiments, the system includes a digital processing device that can control one or more other elements of the system, such as the surface, the sensor, or the emitter. In some embodiments, the digital processing device controls turning on/off of the other elements. In some embodiments, the digital processing device controls sensing, transmitting, or storing the parameter(s). In some embodiments, the digital processing device processes the parameter(s) to generate the guidance. In some embodiments, the digital processing device utilizes the machine learning algorithm to generate the guidance.

In some embodiments, the system includes a digital processing device which can control the surface so that the therapy can be performed automatically, at least in part. In some embodiments, the digital processing device can control the elements with wire or wirelessly.

In some embodiments, the system includes a non-transitory computer readable medium configured to receive the parameter, the signal sensed by the sensor, and outputs a guidance to a therapy provider. In some embodiments, the guidance is to modify, start, or cease a therapy to at least a portion of a body of an individual. In some embodiments, the guidance is to apply more or less pressure to at least a portion of the body. In some embodiments, the guidance is to apply additional therapy to said portion.

In some embodiments, the system herein includes a remote server configured to receive and analyze the parameter, the signal, or any other data communicated to the remote server. In some embodiments, the remote server includes a digital processing device. In some embodiments, the remote server includes a database. In some embodiments, the remote server includes a computer program. In some embodiments, the remote server includes a user interface that allows a user to edit/view functions of the remote server. For example, the user interface allows a user to set a fixed interval, e.g., every 12 hours, for data to be communicated from the sensor(s) to the server to be saved.

In some embodiments, the system herein includes one or more sensors. In some embodiments, the sensors are configured for measuring the same parameter at different locations or different parameters at the same or different locations. A sensor may be configured to measure temperature at one or more locations on a body surface of the individual. A sensor may be configured to measure more than one parameter (such as temperature and an infrared map) at one or more locations on a body surface of the individual. The one or more locations may comprise different locations. The one or more locations may comprise the same location.

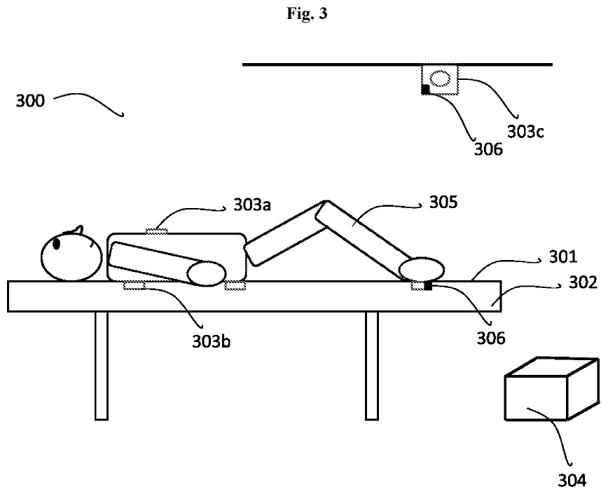

Referring to FIG. 3, in a particular embodiment, the system for providing a therapy to an individual 300 can include a surface 301 that the individual 305 can lie on. The surface 301 can be part of a therapy table 302, chair or use of the same. The system can include one or more sensors 303a, 303b, 303c. In some embodiments, the sensors may be attached to 303a, in contact with 303a, 303b, or remote 303c from the individual. In this particular embodiment, the sensor located remotely from the individual can be an infrared light source, a camera, or any other type of sensors. In some embodiments, one or more sensors in contact with the individual can be a temperature sensor, a heart rate sensor, an EEG sensor, an EMG sensor, a blood sugar sensor, or any other type of sensors.

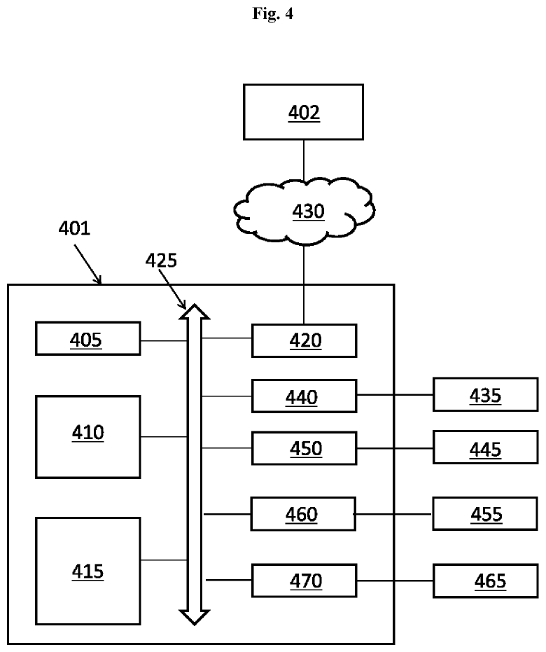

In some embodiments, the sensors 303a, 303b, 303c are powered with a rechargeable power source, e.g., a battery. In some embodiments, the sensors include a communications element 306 that is configured to enable wireless communication of the sensor to a digital processing device 304. An exemplary digital processing device and its components are shown in FIG. 4. In some embodiments, the sensors can be controlled manually by a therapy provider or an individual. In some embodiments, the sensors can be controlled automatically by a remote server comprising a digital processing device. In some embodiments, the sensor can be controlled by a therapy provider application or a therapy recipient application via the remote server.

In some embodiments, the communications element 306 herein includes a transmitter, a receiver, and/or a transceiver. In some embodiments, the receiver, the transmitter, or transceiver is configured to communicate data using one or more wireless data transfer protocols herein. For example, the receiver, the transmitter, or transceiver herein includes a radio transceiver with an antenna or connection for an external antenna for radio frequency signals. In some embodiments, the wireless data transfer protocol includes one or more of Near Field Communication (NFC), wireless USB, ultra-wide-band, ultraband, Wi-Fi, Bluetooth, Bluetooth LE, ZigBee, WiMAX, a radio-wave based protocol, a microwave based protocol, an infrared based protocol, an optical-wave protocol, electromagnetic induction-based protocol, a ultrasonic-wave based protocol, or a sound-wave based protocol.

### Therapy Provider Applications and Therapy Recipient Applications

In some embodiments, the system includes a therapy provider application configured to allow a therapy provider to communicate with the remote server. In some embodiments, the application is a mobile application or a web application.

In some embodiments, the application allows the therapy provider to view/edit information related to a current therapy, existing or future therapy sessions. For example, the therapy provider can monitor one or more parameters of the individual during a therapy session, such as a vital sign of the individual. As another example, the therapy provider can set a vital sign threshold so that the application sends an audio or mechanical signal when the vital sign exceeds the threshold. In some embodiments, the therapy provider can use the application to record the vital sign during the current therapy. In some embodiments, the application allows the therapy provider to control one or more elements of the system. In some embodiments, the application allows the therapy provider to turn on or turn off one or more sensors. In some embodiments, application allows the therapy provider to enter additional information related to the therapy or the individual. For example, the application may allow the therapy provider to input medical history of the individual. As another example, the application may allow the therapy provider to input the individual's description of his or her symptom(s).

In some embodiments, the application may allow the therapy provider to receive a guidance from a remote server or otherwise a digital processing device. In some embodiments, the guidance may include one or more of: an audio signal, a graphical image, a text message, or a combination thereof. In some embodiments, the guidance may include a series of sub-guidance that can be delivered at different time points. In some embodiments, the guidance may be interactive with the therapy provider. For example, the one or more sub-guidance may be altered based on the therapy provider's response or updated inputs related to the individual to optimize the effect of the therapy on the individual.

In some embodiments, the system includes a therapy recipient application configured to communicate with a remote server.

In some embodiments, the therapy recipient application herein allows an individual to view or edit information related to current, existing, and/or future therapies. For example, an individual can view sensed parameter(s) before and after a therapy session to review quantitative effectiveness of the therapy. As another example, the individual may review historical data of the sensed parameter to examine long-term effects of quantitative therapies. In some embodiments, the individual can select a preferred sensor set-up for measuring one or more parameters. For example, a user may select a temperature sensor attached on the surface over a sensor attached to his or her body. In some embodiments, the therapy recipient can enter medical history, symptoms, location of symptoms or other information using the application. In some embodiments, the individual can schedule therapy session(s) using the application.

In some embodiments, disclosed herein is a non-transitory computer readable medium comprising a computer program including instructions that cause a processor to: receive a parameter of an individual sensed with a parameter sensor; and determine a guidance comprising a modification of a therapy delivered by a therapy provider to said individual.

In some embodiments, the parameter comprises a movement of a portion of a body of said individual. In some embodiments, the sensor comprises an infrared light sensor. The infrared light sensor can be operably coupled with an infrared light source that is configured to project an infrared light towards the individual, and wherein the infrared light sensor is configured to sense said infrared light when said infrared light is reflected from the individual.

In some embodiments, the parameter comprises a temperature of a portion of a body of said individual. The parameter can comprise a vital sign of said individual. In some embodiments, the sensor can comprise a thermographic camera, a temperature probe or pad, a heart rate sensor, a blood pressure cuff, an spO₂ sensor, at least one ECG electrode, at least one EEG sensor, and an electroencephalogram (EEG).

In some embodiments, the parameter is sensed in response to a therapy to a first portion of a body of said individual and wherein the guidance comprises indicating to a therapy provider to modify the therapy to the first portion of the body or a second portion of the body.

### Machine Learning Algorithms

In some embodiments, the parameter herein is received as an input to a machine learning algorithm configured to output a guidance. In some embodiments, the machine learning algorithm takes additional input(s) in order to output a guidance. In some embodiments, the additional input(s) include descriptions of symptoms or pain by the individual. In some embodiments, the additional input(s) include medical history of the individual. In some embodiments, the additional input(s) includes a medical professional's description of the individual's problem. In some embodiments, the machine learning algorithm is trained and used to output a guidance when an input is received. In some embodiments, the machine algorithm is used to output a guidance while training can be performed before an input is received, for example, periodically using historical data of the individual and/or a selected group of individuals.

The systems, methods, and media described herein may use machine learning algorithms for training prediction models and/or making predictions of a guidance. Machine learning algorithms herein may learn from and make predictions on data. Data may be any input, intermediate output, previous outputs, or training information, or otherwise any information provided to or by the algorithms.

A machine learning algorithm may use a supervised learning approach. In supervised learning, the algorithm can generate a function or model from training data. The training data can be labeled. The training data may include metadata associated therewith. Each training example of the training data may be a pair consisting of at least an input object and a desired output value. A supervised learning algorithm may require the user to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use an unsupervised learning approach. In unsupervised learning, the algorithm may generate a function/model to describe hidden structures from unlabeled data (i.e., a classification or categorization that cannot be directed, observed, or computed). Since the examples given to the learner are unlabeled, there is no evaluation of the accuracy of the structure that is output by the relevant algorithm. Approaches to unsupervised learning include: clustering, anomaly detection, and neural networks.

A machine learning algorithm may use a semi-supervised learning approach. Semi-supervised learning can combine both labeled and unlabeled data to generate an appropriate function or classifier.

A machine learning algorithm may use a reinforcement learning approach. In reinforcement learning, the algorithm can learn a policy of how to act given an observation of the world. Every action may have some impact in the environment, and the environment can provide feedback that guides the learning algorithm.

A machine learning algorithm may use a transduction approach. Transduction can be similar to supervised learning, but does not explicitly construct a function. Instead, tries to predict new outputs based on training inputs, training outputs, and new inputs.

A machine learning algorithm may use a “learning to learn” approach. In learning to learn, the algorithm can learn its own inductive bias based on previous experience.

A machine learning algorithm is applied to patient data to generate a prediction model. In some embodiments, a machine learning algorithm or model may be trained periodically. In some embodiments, a machine learning algorithm or model may be trained non-periodically.

As used herein, a machine learning algorithm may include learning a function or a model. The mathematical expression of the function or model may or may not be directly computable or observable. The function or model may include one or more parameter(s) used within a model. For example, a linear regression model having a formula Y=C0+C1x1+C2x2 has two predictor variables, x1 and x2, and coefficients or parameter, C0, C1, and C2. The predicted variable in this example is Y. After the parameters of the model are learned, values can be entered for each predictor variable in a model to generate a result for the dependent or predicted variable (e.g., Y).

A machine learning algorithm may comprise a linear regression algorithm, a logistic regression algorithm, a decision tree algorithm, a SVM algorithm, a naive Bayes algorithm, a kNN algorithm, a K-means algorithm, a random forest algorithm, or any combination thereof. A machine learning algorithm may perform supervised learning. A machine learning algorithm may perform unsupervised learning. A machine learning algorithm may perform semi-supervised learning. A machine learning algorithm may perform reinforcement learning.

Methods may include hierarchical clustering. A machine learning algorithm may be trained with a training set. A training set may comprise training data stored in a database. A training set may comprises measured values of one or more parameters, a massage recipe (such as specification for a massage), a therapy recipe, an exercise recipe, or any combination thereof.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display.

In some embodiments, the digital processing device includes an input device to receive information from a user. In some embodiments, the input device is a keyboard. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Referring to FIG. 4, in a particular embodiment, an exemplary digital processing device 401 is programmed or otherwise configured to control sensing, sensing data communication, sensing data processing, and generation of guidance of the systems and methods herein. In this embodiment, the digital processing device 401 includes a central processing unit (CPU, also “processor” and “computer processor” herein) 405, which can be a single core or multi core processor, or a plurality of processors for parallel processing. The digital processing device 401 also includes memory or memory location 410 (e.g., random-access memory, read-only memory, flash memory), electronic storage unit 415 (e.g., hard disk), communication interface 420 (e.g., network adapter, network interface) for communicating with one or more other systems, and peripheral devices, such as cache, other memory, data storage and/or electronic display adapters. The peripheral devices can include storage device(s) or storage medium 465 which communicate with the rest of the device via a storage interface 470. The memory 410, storage unit 415, interface 420 and peripheral devices are in communication with the CPU 405 through a communication bus 425, such as a motherboard. The storage unit 415 can be a data storage unit (or data repository) for storing data. The digital processing device 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the device 401, can implement a peer-to-peer network, which can enable devices coupled to the device 401 to behave as a client or a server.

Continuing to refer to FIG. 4, the digital processing device 401 includes input device(s) 445 to receive information from a user, the input device(s) in communication with other elements of the device via an input interface 450. The digital processing device 401 can include output device(s) 455 that communicates to other elements of the device via an output interface 460.

Continuing to refer to FIG. 4, the memory 410 can include various components (e.g., machine readable media) including, but not limited to, a random-access memory component (e.g., RAM) (e.g., a static RAM “SRAM”, a dynamic RAM “DRAM, etc.), or a read-only component (e.g., ROM). The memory 410 can also include a basic input/output system (BIOS), including basic routines that help to transfer information between elements within the digital processing device, such as during device start-up, can be stored in the memory 410.

Continuing to refer to FIG. 4, the CPU 405 can execute a sequence of machine-readable instructions, which can be embodied in a program or software. The instructions can be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and write back. The CPU 405 can be part of a circuit, such as an integrated circuit. One or more other components of the device 401 can be included in the circuit. In some embodiments, the circuit is an application specific integrated circuit (ASIC) or a field programmable gate array (FPGA).

Continuing to refer to FIG. 4, the storage unit 415 can store files, such as drivers, libraries and saved programs. The storage unit 415 can store user data, e.g., user preferences and user programs. The digital processing device 401 in some cases can include one or more additional data storage units that are external, such as located on a remote server that is in communication through an intranet or the Internet. The storage unit 415 can also be used to store operating systems, application programs, and the like. Optionally, storage unit 415 can be removably interfaced with the digital processing device (e.g., via an external port connector (not shown)) and/or via a storage unit interface. Software may reside, completely or partially, within a computer-readable storage medium within or outside of the storage unit 415. In another example, software may reside, completely or partially, within processor(s) 405.

Continuing to refer to FIG. 4, the digital processing device 401 can communicate with one or more remote computer systems 402 through the network 430. For instance, the device 401 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. In some embodiments, the remote computer system is configured for image and signal processing of images acquired using the image systems herein. In some embodiments, the imaging systems herein allows partitioning of image and signal processing between a processor in the imaging head (e.g. based on a MCU, DSP or FPGA) and a remote computer system, i.e., a back-end server.

Continuing to refer to FIG. 4, information and data can be displayed to a user through a display 435. The display is connected to the bus 425 via an interface 440, and transport of data between the display other elements of the device 401 can be controlled via the interface 440.

Methods as described herein can be implemented by way of machine (e.g., computer processor) executable code stored on an electronic storage location of the digital processing device 401, such as, for example, on the memory 410 or electronic storage unit 415. The machine executable or machine-readable code can be provided in the form of software. During use, the code can be executed by the processor 405. In some embodiments, the code can be retrieved from the storage unit 415 and stored on the memory 410 for ready access by the processor 405. In some situations, the electronic storage unit 415 can be precluded, and machine-executable instructions are stored on memory 410.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some embodiments, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight Java™, and Unity®.

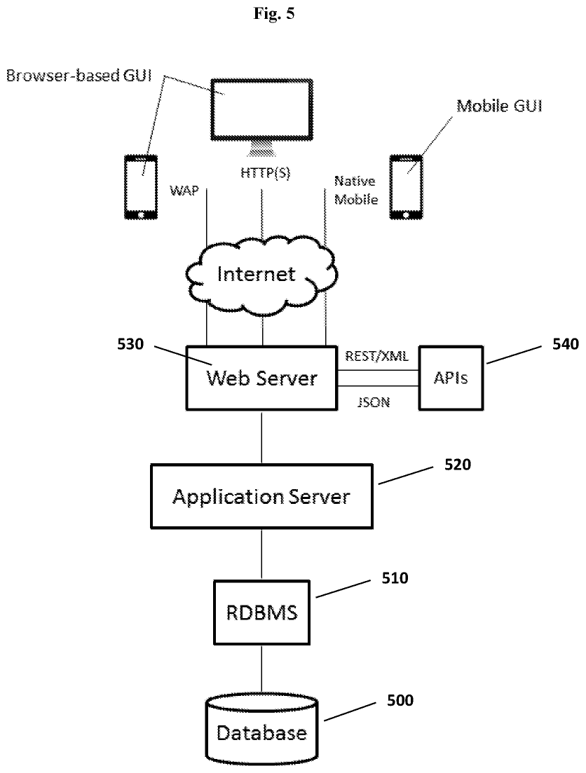

Referring to FIG. 5, in a particular embodiment, an application provision system comprises one or more databases 500 accessed by a relational database management system (RDBMS) 510. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 520 (such as Java servers, .NET servers, PHP servers, and the like) and one or more web servers 530 (such as Apache, IIS, GWS and the like). The web server(s) optionally expose one or more web services via app application programming interfaces (APIs) 540. Via a network, such as the Internet, the system provides browser-based and/or mobile native user interfaces.

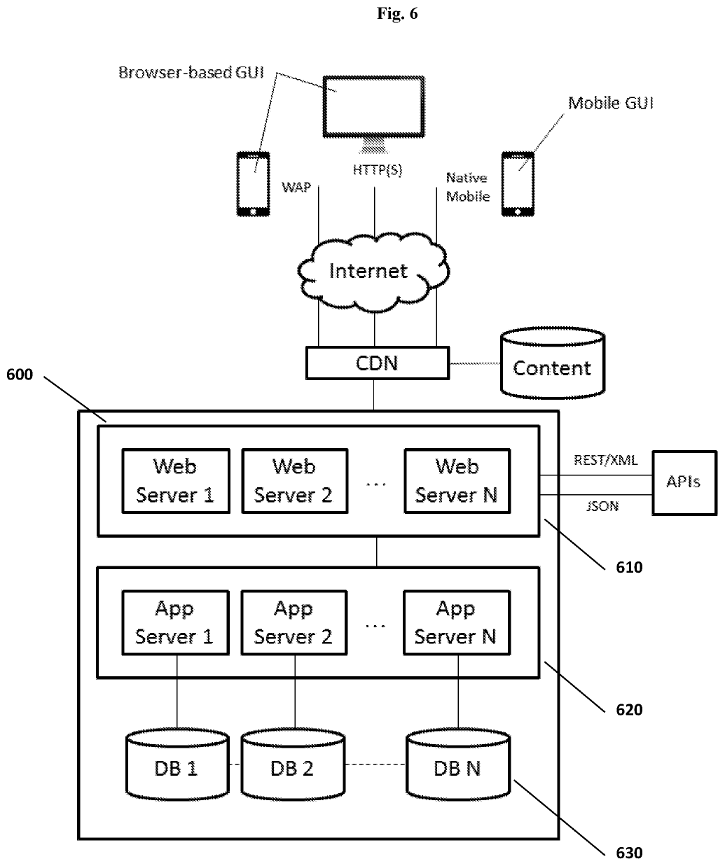

Referring to FIG. 6, in a particular embodiment, an application provision system alternatively has a distributed, cloud-based architecture 600 and comprises elastically load balanced, auto-scaling web server resources 610 and application server resources 620 as well synchronously replicated databases 630.

In some embodiments, a system can include a display (such as a graphical user interface, an iPad, an iPod, a cellular phone, a computer screen, a television screen) that may be coupled to (a) a computer (such as a portable computer), (b) an energy source (such as a battery, a rechargeable battery, a non-rechargeable battery, a solar panel, or an electrical outlet), (c) an imaging device (such as a digital camera, a CCD camera), or (d) a combination of any of these. Components of the system may be coupled and decoupled from one another. For example, an energy source may be coupled to a display during a charging period and the display may be decoupled from the energy source during use. An imaging device may be coupled to a display and decoupled from the display.

An imaging device (such as a digital camera) may be integral to the display, may be separate from the display, or may be culpable to the display. An imaging device can be hand-held. An imaging device can be portable. An imaging device can be fixed, such as temporarily fixed in a position or temporarily fixed onto another component of the system. An imaging device can be fixed (such as temporarily fixed) to another component of the system, such as a portion of a cart (such as an arm or beam) or a surface of a system element (such as a therapy table).

A position of an imaging device may be adjustable. A direction of image collection of the imaging device may be adjustable. For example, an imaging device may maintain a position but may adjust a direction of image collection. An imaging device may adjust a position and a direction of image collection at a same time or a different time. A position or direction of image collection may be adjusted in any direction, such as vertically, horizontally, diagonally, or any combination thereof. One or more images collected or detected by the imaging device can be collected or detected in a direct orientation, an oblique orientation, a side orientation, or any combination thereof.

A distance from an imaging device to an individual may be adjustable. For example, a distance from an imaging device to an individual may be increased or decreased. A distance may be adjusted discreetly, continuously, automatically, manually, or any combination thereof. A distance may be adjusted prior to acquisition of one or more images. A distance may be adjusted during a procedure, such as a therapy. A distance may be adjusted based on a parameter such as an individual parameter (such as an individual's height or individual's orientation such as laying down or standing), a procedure type, an imaging device type, or any combination thereof. A distance from an imaging device to an individual may be about: 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150 inches or more. A distance from an imaging device to an individual may be about 20 inches. A distance from an imaging device to an individual may be about 30 inches. A distance from an imaging device to an individual may be about 40 inches. A distance from an imaging device to an individual may be about 50 inches. A distance from an imaging device to an individual may be about 60 inches. A distance from an imaging device to an individual may be about 70 inches. A distance from an imaging device to an individual may be about 80 inches. A distance from an imaging device to an individual may be about 90 inches. A distance from an imaging device to an individual may be about 100 inches. A distance from an imaging device to an individual may be from about 10 inches to about 100 inches. A distance from an imaging device to an individual may be from about 10 inches to about 80 inches. A distance from an imaging device to an individual may be from about 20 inches to about 100 inches. A distance from an imaging device to an individual may be from about 20 inches to about 100 inches. A distance from an imaging device to an individual may be adjustable from about 10 inches to about 150 inches. A distance from an imaging device to an individual may be adjustable from about 10 inches to about 100 inches. A distance from an imaging device to an individual may be from about 1 inch to about 150 inches. A distance from an imaging device to an individual may be from about 1 inch to about 100 inches. A distance from an imaging device to an individual may be from about 1 inch to about 50 inches. A distance from an imaging device to an individual may be from about 10 inches to about 50 inches. A distance from an imaging device to an individual may be from about 10 inches to about 100 inches. A distance from an imaging device to an individual may be from about 25 inches to about 100 inches. A distance from an imaging device to an individual may be from about 25 inches to about 150 inches. A distance from an imaging device to an individual may be from about 25 inches to about 200 inches. A distance may be measured from a surface of an individual such as a skin surface. A distance may be measured from a location on an individual such as a head portion, a chest portion, a back portion, a leg portion, an arm portion or any other location on an individual. A distance may be measured from an external surface of the imaging device. A distance may be measured from a capture source of the imaging device.

A position of an imaging device may be adjustable relative to a position of an individual. For example, an imaging device may be positioned in front of, behind, to the left of, to the right of, above, below the individual or any combination of relative positions thereof. An imaging device may be programmed to adjust position relative to an individual automatically. A position of an imaging device may be manually adjusted relative to an individual. A position of an imaging device may translate from position x1, y1, z1 to position x2, y2, z2 during a procedure on the individual, such as a therapy that the individual may receive. A position of an imaging device relative to a position of an individual may be specified by a procedure. Previous positions of an imaging device or distance from an imaging device to an individual may be stored in a database that may be part of or separate from the system.

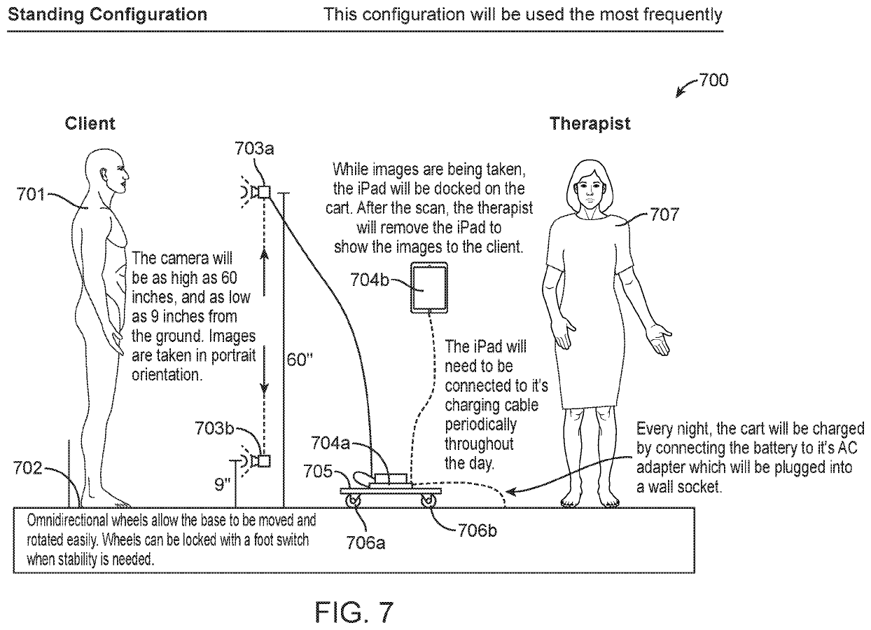

Referring to FIG. 7, this figure shows a standing configuration 700 where an individual 701 is in a standing position on a surface 702. A second individual 707, such as a therapist or technician may utilize the system to acquire one or more images from the individual 701. A camera position may be adjustable relative to the individual 701, such as adjusting for a subject's height or a portion of a subject's body to be imaged. A camera position may be adjusted relative to a surface upon which the individual is standing. For example, in some embodiments, a camera may be positioned from about 9 inches 703b to about 60 inches 703a from a surface. An image or data obtained from the camera may be acquired in a portrait orientation, a landscape orientation, or a combination thereof.

An element, such as one or more omnidirectional wheels (706a and 706b) may be coupled to a base element 705 to allow the base element to be moved and rotated easily. The movable element (such as wheels) can be locked with a locking element (such as a foot switch), such as when stability is needed or when the base element is needed in a fixed position.

In some embodiments, (such as when images are being taken), the display 704a (such as an iPad) may be docked on a base element, such as a cart. In some embodiments, the display 704b may be removed from the base element, such as after an imaging such that a second individual (i.e., Therapist) can share one or more images to the individual (client). The display may be coupled and decoupled from an energy source, such as a charging cable. In some embodiments, the cart or base element comprising the display may be coupled to an energy source. For example, a cart may be charged by connecting the battery to its AC adapter which may be plugged into a wall socket.

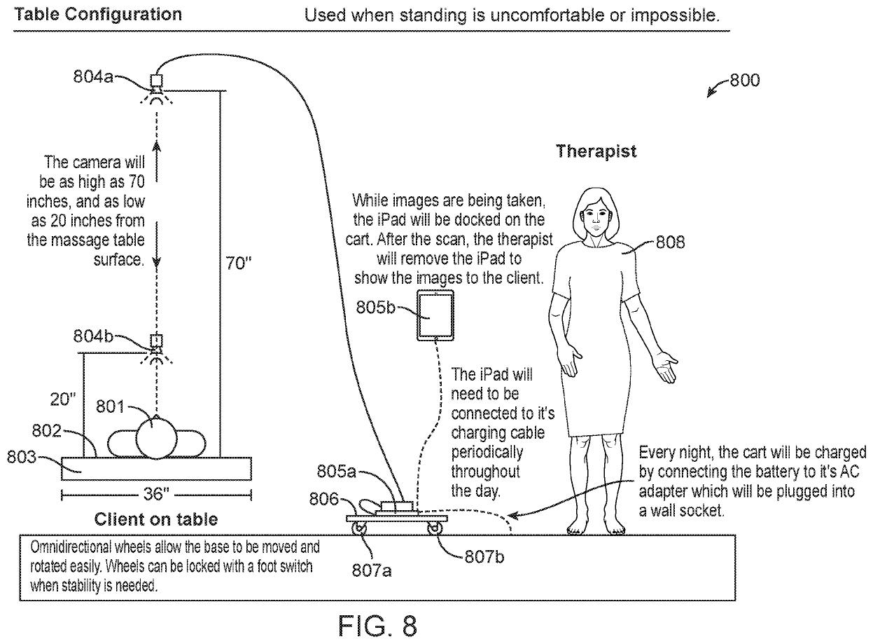

Referring to FIG. 8, this figure shows a table configuration 800. This configuration may be employed when an individual 801 has difficulty standing or cannot stand. The individual 801 may be positioned on a surface 802 of an element 803 (such as a massage table), such as laying a back side of the individual onto the surface 802. A position of a camera may be adjusted relative to the individual 801. For example, in some embodiments, a camera may be positioned from about 20 inches 804a to about 70 inches 804b from a surface of the individual.

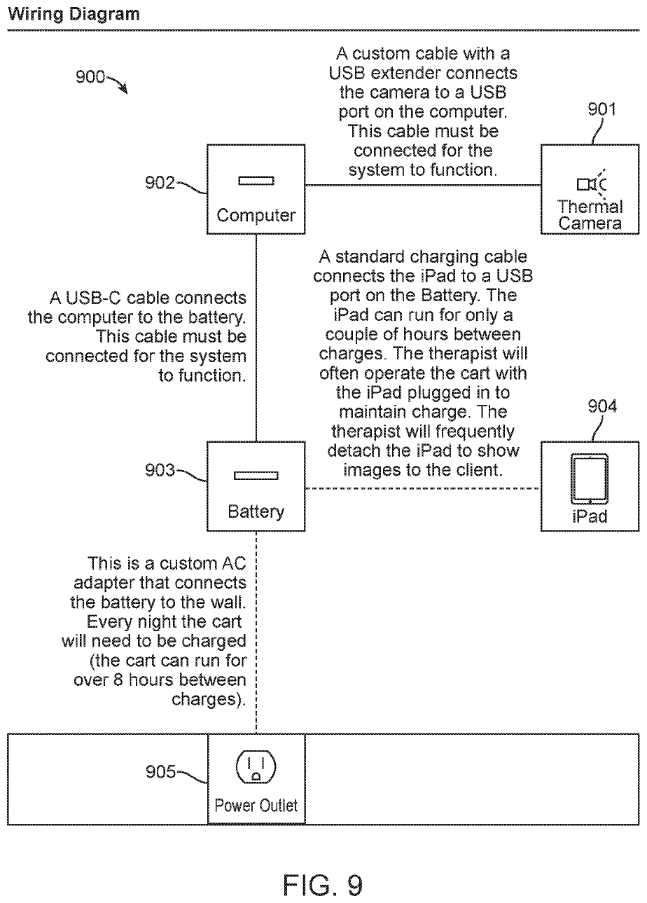

An element, such as one or more omnidirectional wheels (807a and 807b) may be coupled to a base element 806 to allow the base element to be moved and rotated easily. The movable element (such as wheels) can be locked with a locking element (such as a foot switch), such as when stability is needed or when the base element is needed in a fixed position. In some embodiments, (such as when images are being taken), the display 804b (such as an iPad) may be docked on a base element, such as a cart. In some embodiments, the display 805b may be removed from the base element, such as after an imaging such that a second individual 808 (i.e., therapist or technician) can share images to the individual (client). The display may be coupled and decoupled from an energy source, such as a charging cable. In some embodiments, the cart or base element comprising the display may be coupled to an energy source. For example, a cart may be charged by connecting the battery to its AC adapter which may be plugged into a wall socket. Referring to FIG. 9, this figure shows a wiring diagram 900. An imaging device 901 (such as a digital camera or thermal camera) may be coupled to a computer 902, an energy source 903 (such as a battery), a display 904 (such as an iPad), a power outlet 905, or any combination thereof. In some embodiments, a cable (such as a custom cable with a USB extender) may connect the imaging device (i.e., camera) to a USB port on the computer. This cable may be connected for the system to function. In some embodiments, a cable (such as a USB-C cable) may connect the computer to the energy source (i.e., battery). This cable may be connected for the system to function. A cable (such as a standard charging cable) may connect the display (such as an iPad) to the USB port on the energy source (i.e., battery). The display (iPad) may operate for one or more hours between charges. The second individual (such as a therapist or technician) may operate the cart with the display (i.e., iPad) plugged in to maintain charge. The second individual (i.e., therapist or technician) may decouple the display (iPad) from the cart to show one or more images to the individual (i.e., client). In some embodiments, an adapter (such as a custom AC adapter) may connect the battery to the wall power outlet. One or more components on a cart may need to be charged one or more times a day. For example, a display (iPad) may run for about: 4, 5, 6, 7, 8, 9, 10, 11, 12, 13, 14, 15 hours between charges.

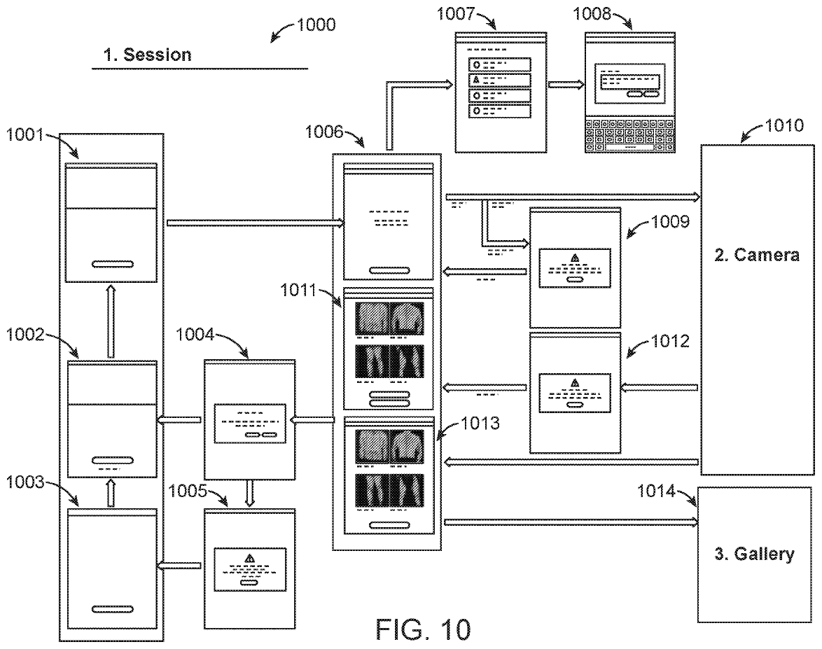

Referring to FIG. 10, this figure shows a flow diagram of a session 1000. A user can select a ‘begin’ tab to start a session 1001. A user can select a ‘take pictures’ tab on the display 1006 to start acquiring one or more images. The display may show one or more settings 1007, such as active thermal camera settings. A user can select to edit the camera settings 1008. A user can select a camera. A connection to the camera may be successful. A connection to the camera may fail 1009 or 1012. If the connection fails, the user can adjust one or more camera settings or adjust the internet connection. If the connection is successful, a user can acquire one or more images using the camera 1010. When a user completes acquisition of one of more images, a user may select a ‘session’ tab on the camera screen or may be guided to prepare a report 1013 or 1011. A report may include one or more images, such as one or more images collected at different times or one or more images collected from different locations of an individual's body. Images may be selected to be included in a report. A report may be selected to be submitted to a photo gallery 1014. Selecting an image may load the image full-screen in a photo gallery 1014. When a session is complete, a user can select ‘end’ session 1004. Images may be transferred to a server. When successfully transferred, a user can select ‘OK’ and begin a new session 1002. When an upload to a server includes an error 1005, a user can select ‘OK’ and begin a new session 1003.

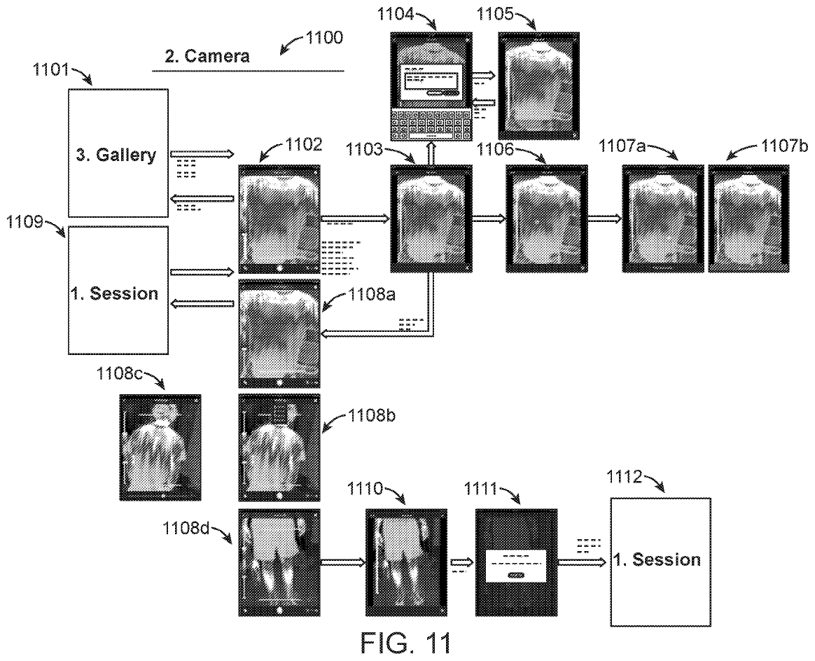

FIG. 11 shows a flow diagram of a camera 1100. This session 1109 may begin when a user launches a camera to show a live image 1108a. When a user may select a ‘gallery’ tab, the display may re-direct to a gallery 1101. When a user selects a ‘back’ tab, the display may return to a live image 1102 from the camera (such as when a user may decide to retake a photo). When a user selects a ‘report’ tab, the display may return to the session 1109. A user may acquire one or more images 1108a, 1108b, 1108c, 1108d. When a user has completed taking one or more images 1110, a user may select to complete a session 1111. A user may select to review a session 1112, a report, or both on the display. A user may acquire one or more images. An overlay for a subsequent image may be activated when a camera launches. If one or more images have been acquired, a camera may launch without an overlay and one or more additional images may be taken. When a user selects an ‘update’ tab, the display may be updated to a live image for acquiring a next image. A user may select one or more images 1103 to add a note to one or more images 1104. When a ‘done’ tab is selected, the note may be added to the image 1105. When a ‘note’ icon is selected, the user may update the note. A user may select one or more images 1106. A user may annotate one or more images, such as dragging at least a selected portion of an image 1107a, 1107b.

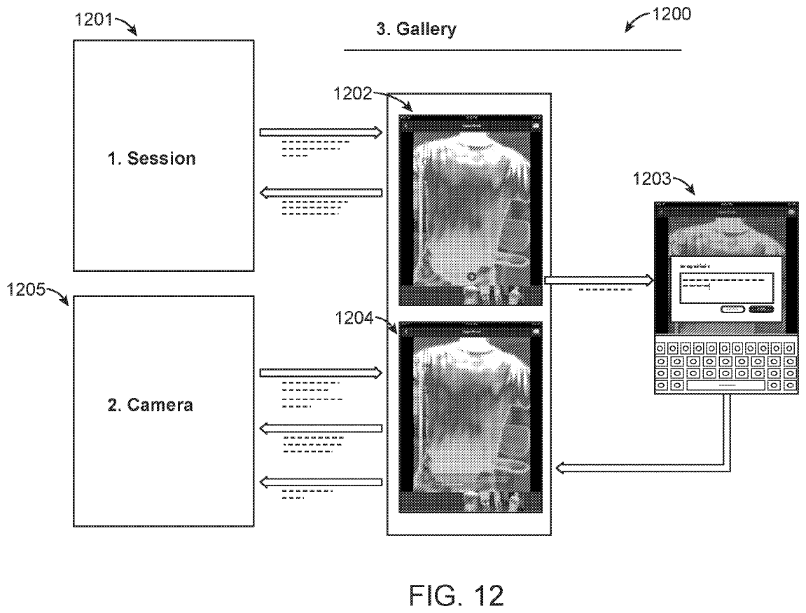

FIG. 12 shows a flow diagram of a gallery 1200. This session 1201 may begin by a user selecting one or more images in a report to load to the display 1202. A user can select a ‘back’ button to the start of the session (such as when a gallery has been launched from a session). A user may select a ‘note’ icon to add notes or a description to one or more images 1203. A user may select a ‘done’ button when the notes or description are complete. A user may select to retake an image using the camera. A user may select a back button (such as when a gallery has been launched from a camera). When a user selects the gallery icon from the camera 1205, a most recently acquired image 1204 may be loaded.

In some embodiments, the system may provide a feature (such as a visual feature displayed on the display) for an individual (i.e., practitioner or therapist) to view one or more images collected over a period of time. The feature may be executed with the use of a slide bar on the display that the individual can manipulate by sliding to visualizing the collection of images in a time step manner or sequential based on an image parameter such as time stamp, image type, or other. This feature may permit an individual to review previous images, to review progress or changes shown in the collection of previous images, to aid in decision making of a current therapeutic or massage session. In some embodiments, at least about 20 images can be viewed with a slide bar feature. In some embodiments, at least about 50 images can be viewed. In some embodiments, at least about 100 images can be viewed. In some embodiments, at least about 200 images can be viewed. In some embodiments, at least from about 1 image to about 200 images can be viewed. In some embodiments, at least from about 10 images to about 500 images can be viewed. In some embodiments, at least from about 1 image to about 100 images can be viewed. In some embodiments, at least from about 1 image to about 50 images can be viewed. In some embodiments, at least from about 10 images to about 200 images can be viewed. In some embodiments, at least a portion of images taken within about a 1 year time period can be viewed. In some embodiments, at least a portion of images taken within about a 2 year time period can be viewed. In some embodiments, at least a portion of images taken within about a 5 year time period can be viewed. In some embodiments, at least a portion of images taken within from about a 1 year to about a 5 year time period can be viewed. In some embodiments, at least a portion of images taken within from about a 1 year to about a 2 year time period can be viewed. In some embodiments, at least a portion of images taken within from about a 1 year to about a 10 year time period can be viewed.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of massage record, information of an individual, sensing data, and/or generated guidance. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. A method for providing a massage to an individual, said method comprising:
(a) sensing a parameter associated with said individual in response to said massage using a sensor; and
(b) guiding said massage based on said parameter.

2. The method of claim 1, wherein said parameter comprises a movement of a portion of a body of said individual.

3. The method of claim 2, wherein said sensor comprises an infrared light sensor.

4. The method of claim 3, wherein said infrared light sensor is operably coupled with an infrared light source that is configured to project an infrared light towards the individual, and wherein the infrared light sensor is configured to sense said infrared light when said infrared light is reflected from the individual.

5. The method of claim 1, wherein said parameter comprises a temperature of a portion of a body of said individual or a vital sign of said individual.

6. The method of claim 5, wherein said sensor comprises a thermographic camera, a temperature probe or pad, a heart rate sensor, a blood pressure cuff, an spO₂ sensor, an ECG electrode, or an EEG sensor.

7. The method of claim 1, wherein said parameter comprises an electrocardiogram (ECG) or an electroencephalogram (EEG) of said individual.

8. The method of claim 1, wherein step (b) comprises indicating to a massage provider to modify said massage.

9. The method of claim 1, wherein said parameter is sensed in response to said massage to a first portion of a body of said individual and wherein step (b) comprises indicating to a massage provider to modify said massage to said first portion of said body.

10. The method of claim 9, wherein indicating to a massage provider to modify said massage to said first portion of said body comprises indicating to said massage provider to apply more pressure to said first portion of said body, apply less pressure to said first portion of said body, apply additional massage to said first portion of said body, or cease massage to said first portion of said body.

11. The method of claim 1, wherein said parameter is sensed in response to said massage to a first portion of a body of said individual and wherein said parameter relates to said first portion of said body.

12. The method of claim 11, wherein said parameter comprises a movement of said first portion of said body of said individual or a thermal reading of said first portion of said body.

13. The method of claim 1, wherein said parameter is sensed in response to said massage to a first portion of a body of said individual and wherein said parameter comprises at least one of: a respiration rate, an oxygenation, a heart rate, a heart rhythm, a blood pressure, or a brain function.

14. The method of claim 1, wherein said parameter is sensed in response to said massage to a first portion of a body of said individual and wherein step (b) comprises indicating to a massage provider to modify said massage to a second portion of said body.

15. The method of claim 14, wherein indicating to a massage provider to modify said massage to said second portion of said body comprises indicating to said massage provider to apply more pressure to said second portion of said body, apply less pressure to said first portion of said body, apply additional massage to said first portion of said body, or cease massage to said first portion of said body.

16. The method of claim 1, wherein said parameter is sensed in response to said massage to a first portion of a body of said individual and wherein said parameter relates to a second portion of said body.

17. The method of claim 1, wherein said parameter is received as an input to a machine learning algorithm configured to output a guidance.

18. The method of claim 17, wherein step (b) comprises presenting said guidance to a massage provider.

19. A system for use in providing a massage to an individual, said system comprising:
(a) a surface upon which said individual lies or sits;
(b) a first sensor coupled to said surface and configured to sense a parameter associated with said individual when said individual lies or sits on said surface; and
(c) a second sensor positioned to sense a signal emitted by or reflected by a portion of said individual when said individual lies or sits on said surface.

20. The system of claim 19, wherein said surface comprises a massage table or a massage chair.

21. The system of claim 19, wherein said first sensor is positioned on said surface so that it contacts said individual when said individual lies or sits on said surface.

22. The system of claim 19, wherein said first sensor comprises a vital sign sensor, a heart rate sensor, a blood pressure sensor, an spO₂ sensor, an ECG sensor, an EEG sensor, or an infrared sensor.

23. The system of claim 22, wherein said infrared sensor is a component of a thermogenic camera.

24. The system of claim 19, wherein said second sensor is configured to detect motion or sense energy emitted or reflected from the individual.

25. The system of claim 24, comprising at least one of: an infrared emitter, an ultrasound emitter, or a radiofrequency emitter.

26. The system of claim 19, comprising a non-transitory computer readable medium configured to receive said parameter and said signal and output a guidance to a massage provider.

27. The system of claim 26, wherein said guidance is to modify a massage to a portion of a body of an individual.

28. The system of claim 27, wherein said guidance is to apply more pressure to said portion, apply less pressure to said portion, apply additional massage to said portion, or cease massage to said first portion.

29. The system of claim 19, comprising a remote server configured to receive and analyze said parameter and said signal as well as a massage provider application configured to communicate with said remote server.

30. The system of claim 29, comprising a massage recipient application configured to communicate with said remote server.

31. A non-transitory computer readable medium comprising a computer program including instructions that cause a processor to:
(a) receive a parameter of an individual sensed with a parameter sensor; and
(b) determine a guidance comprising a modification of a massage delivered by a massage provider to said individual.

32. The medium of claim 31, wherein said parameter comprises a movement or a temperature of a portion of a body of said individual.

33. The medium of claim 32, wherein said sensor comprises an infrared light sensor, a thermographic camera, a temperature probe or pad, a heart rate sensor, a blood pressure cuff, an spO₂ sensor, an ECG electrode, or an EEG sensor.

34. The medium of claim 33, wherein said infrared light sensor is operably coupled with an infrared light source that is configured to project an infrared light towards the individual, and wherein the infrared light sensor is configured to sense said infrared light when said infrared light is reflected from the individual.

35. The medium of claim 31, wherein said parameter comprises an electrocardiogram (ECG) or an electroencephalogram (EEG) of said individual.

36. The medium of claim 31, wherein said parameter is sensed in response to said massage to a first portion of a body of said individual and wherein said guidance comprises indicating to a massage provider to modify said massage to said first portion of said body.

37. The medium of claim 31, wherein said parameter is sensed in response to said massage to a first portion of a body of said individual and wherein said guidance relates to said first portion of said body.

38. The medium of claim 31, wherein said parameter is sensed in response to said massage to a first portion of a body of said individual and wherein said parameter comprises at least one of: a respiration rate, an oxygenation, a heart rate, a heart rhythm, a blood pressure, or a brain function.

39. The medium of claim 31, wherein said parameter is sensed in response to said massage to a first portion of a body of said individual and wherein said guidance comprises indicating to a massage provider to modify said massage to a second portion of said body.

40. A method for guiding meditation, said method comprising:
(c) sensing a parameter associated with said individual while said individual is meditating using a sensor; and
(d) providing guidance to said individual based on said parameter.
